(19) United States
(12) Patent Application Publication
(10) Pub. No.: US 2013/0244294 A1
(43) Pub. Date: Sep. 19, 2013

(54) PROCESSING BIOMASS

(71) Applicant: XYLECO, INC., Woburn, MA (US)

(72) Inventors: Marshall MEDOFF, Brookline, MA (US); Thomas Craig MASTERMAN, Brookline, MA (US)

(73) Assignee: XYLECO, INC., Woburn, MA (US)

(21) Appl. No.: 13/888,543

(22) Filed: May 7, 2013

Related U.S. Application Data
(63) Continuation of application No. 13/276,192, filed on Oct. 18, 2011.
(60) Provisional application No. 61/394,851, filed on Oct. 20, 2010.

(51) Int. Cl. C12P 19/14

(52) U.S. Cl. CPC C12P 19/14
USPC 435/99; 435/158; 435/162; 435/157; 435/160; 435/134; 435/140; 435/144; 435/110; 435/139; 435/166; 435/71.1; 435/209; 435/135; 435/148; 435/147; 435/141; 435/146

(57) ABSTRACT

Methods of manufacturing fuels are provided. These methods use often difficult to process lignocellulosic materials, for example crop residues and grasses. The methods can be readily practiced on a commercial scale in an economically viable manner, in some cases using as feedstocks materials that would otherwise be discarded as waste.

### CROSS REFERENCE TO RELATED APPLICATIONS

This application is a Continuation of co-pending U.S. Ser. No. 13/276,192 filed Oct. 18, 2011, which claimed priority to U.S. Provisional Application Ser. No. 61/394,851, filed Oct. 20, 2010. The entirety of each of these applications is incorporated herein by reference.

### BACKGROUND

Cellulosic and lignocellulosic materials are produced, processed, and used in large quantities in a number of applications. Often such materials are used once, and then discarded as waste, or are simply considered to be waste materials, e.g., sewage, bagasse, sawdust, and stover.

### SUMMARY

Generally, this invention relates to methods of manufacturing fuels and other products using biomass, e.g., cellulosic and lignocellulosic materials, and in particular often difficult-to-process lignocellulosic materials, for example crop residues and grasses. The methods disclosed herein can be readily practiced on a commercial scale in an economically viable manner, in some cases using as feedstocks materials that would otherwise be discarded as waste.

The methods disclosed herein feature enhancements to four aspects of material processing: (1) mechanical treatment of the feedstock, (2) reduction of the recalcitrance of the feedstock by irradiation, (3) conversion of the irradiated feedstock to sugars by saccharification, and (4) fermentation of the sugars to convert the sugars to other products, such as a solid, liquid, or gaseous fuel, e.g., a combustible fuel, or any of the other products described herein, e.g., an alcohol, such as ethanol, isobutanol, or n-butanol, a sugar alcohol, such as erythritol, an organic acid, e.g., an amino acid, citric acid, lactic acid, or glutamic acid, or mixtures thereof. Combining two or more of the enhancements described herein, in any combination, can in some cases further enhance processing.

In some implementations, the methods disclosed herein include treating a cellulosic or lignocellulosic material to alter the structure of the material by irradiating the material with relatively low voltage, high power electron beam radiation.

In one aspect, the invention features a method that includes irradiating a cellulosic or lignocellulosic material with an electron beam operating at a voltage of less than 3 MeV, e.g., less than 2 MeV, less than 1 MeV, or 0.8 MeV or less and a power of at least 25 kW, e.g., at least 30, 40, 50, 60, 65, 70, 80, 100, 125, or 150 kW, and combining the irradiated cellulosic or lignocellulosic material with an enzyme and/or a microorganism, the enzyme and/or microorganism utilizing the irradiated cellulosic or lignocellulosic material to produce a solid, liquid or gaseous fuel or other product, e.g., an alcohol, such as ethanol, isobutanol, or n-butanol, a sugar alcohol, such as erythritol, or an organic acid.

Some implementations include one or more of the following features. The method can further include soaking the irradiated cellulosic or lignocellulosic material in water at a temperature of at least 40° C., e.g., 60-70° C., 70-80° C. or 90-95° C., prior to combining the irradiated cellulosic or lignocellulosic material with the enzyme and/or microorganism. Irradiating can be performed at a dose rate of at least 0.5 Mrad/sec. The cellulosic or lignocellulosic material can, for example, include corncobs, or a mixture of corncobs, corn kernels and corn stalks. In some cases the material includes entire corn plants.

In another aspect, the invention features a method that includes irradiating a cellulosic or lignocellulosic material with an electron beam, soaking the irradiated cellulosic or lignocellulosic material in water at a temperature of at least 40° C., and combining the irradiated cellulosic or lignocellulosic material with an enzyme and/or a microorganism, the enzyme and/or microorganism utilizing the irradiated cellulosic or lignocellulosic material to produce a fuel or other product, e.g., an alcohol, such as ethanol, isobutanol, or n-butanol, a sugar alcohol, such as erythritol, or an organic acid.

Some implementations include one or more of the following features. In some cases, the electron beam operates at a voltage of less than 3 MeV, e.g., less than 2 MeV or less than 1 MeV, and a power of at least 25 kW, e.g., at least 30, 40, 50, 60, 65, 70, 80, 100, 125, or 150 kW. Irradiating can be performed at a dose rate of at least 0.5 Mrad/sec. The cellulosic or lignocellulosic material can, for example, include corncobs, or a mixture of corncobs, corn kernels and corn stalks. In some cases the material includes entire corn plants.

In another aspect, the invention features a method that includes irradiating a cellulosic or lignocellulosic material with an electron beam at a dose rate of at least 0.5 Mrad/sec, the electron beam operating at a voltage of less than 1.0 MeV, and combining the irradiated cellulosic or lignocellulosic material with an enzyme and/or a microorganism, the enzyme and/or microorganism utilizing the irradiated cellulosic or lignocellulosic material to produce a fuel or other product, e.g., an alcohol, such as ethanol, isobutanol, or n-butanol, a sugar alcohol, such as erythritol, or an organic acid.

Some implementations include one or more of the following features. The method can further include soaking the irradiated cellulosic or lignocellulosic material in water at a temperature of at least 40° C., e.g., 60-70° C., 70-80° C. or 90-95° C., prior to combining the irradiated cellulosic or lignocellulosic material with the enzyme and/or microorganism. In some cases, the electron beam operates at a power of at least 25 kW, e.g., at least 30, 40, 50, 60, 65, 70, 80, 100, 125, or 150 kW. The cellulosic or lignocellulosic material can, for example, include corncobs, or a mixture of corncobs, corn kernels and corn stalks. In some cases the material includes entire corn plants.

In a further aspect, the invention features a method that includes irradiating a cellulosic or lignocellulosic material with an electron beam, the cellulosic or lignocellulosic material comprising corn cobs, corn kernels, and corn stalks, and combining the irradiated cellulosic or lignocellulosic material with an enzyme and/or a microorganism, the enzyme and/or microorganism utilizing the irradiated cellulosic or lignocellulosic material to produce a fuel or other product, e.g., an alcohol, such as ethanol, isobutanol, or n-butanol, a sugar alcohol, such as erythritol, or an organic acid.

Some implementations include one or more of the following features. The method can further include soaking the irradiated cellulosic or lignocellulosic material in water at a temperature of at least 40° C., e.g., 60-70° C., 70-80° C. or 90-95° C., prior to combining the irradiated cellulosic or lignocellulosic material with the enzyme and/or microorganism. In some cases, the electron beam operates at a voltage of less than 3 MeV, e.g., less than 2 MeV or less than 1 MeV, and a power of at least 25 kW, e.g., at least 30, 40, 50, 60, 65, 70, 80, 100, 125, or 150 kW. Irradiating can be performed at a dose rate of at least 0.5 Mrad/sec. In some cases the material includes entire corn plants, and the method further includes obtaining the cellulosic or lignocellulosic material by harvesting entire corn plants.

In yet another aspect, the invention features a method that includes irradiating a cellulosic or lignocellulosic material at a dose rate of at least 0.5 Mrad/sec, with an electron beam operating a voltage of less than 3 MeV, e.g., less than 2 MeV or less than 1 MeV, and a power of at least 25 kW, e.g., at least 30, 40, 50, 60, 65, 70, 80, 100, 125, or 150 kW, transferring the irradiated cellulosic or lignocellulosic material to a tank, and dispersing the cellulosic or lignocellulosic material in an aqueous medium in the tank, and saccharifying the irradiated cellulosic or lignocellulosic material, while agitating the contents of the tank with a jet mixer.

Some implementations include one or more of the following features. The method can further include, after saccharification, isolating sugars from the contents of the tank, and/or fermenting the contents of the tank, in some cases without removing the contents from the tank, to produce a fuel or other product, e.g., an alcohol, such as ethanol, isobutanol, or n-butanol, a sugar alcohol, such as erythritol, or an organic acid. The method can further include hammermilling the cellulosic or lignocellulosic material prior to irradiating. The cellulosic or lignocellulosic material can include corncobs. Irradiating can include delivering to the cellulosic or lignocellulosic material a total dose of from about 25 to 35 Mrads. Irradiating can in some cases include multiple passes of irradiation, each pass delivering a dose of 20 Mrads or less, e.g., 10 Mrads or less, or 5 Mrads or less. The method may further include soaking the irradiated cellulosic or lignocellulosic material in water at a temperature of at least 40° C. prior to combining the irradiated cellulosic or lignocellulosic material with the microorganism.

In a further aspect, the invention features a method comprising irradiating a lignocellulosic material with an electron beam, the lignocellulosic material comprising corn cobs and having a particle size of less than 1 mm, and combining the irradiated lignocellulosic material with an enzyme and/or a microorganism, the enzyme and/or microorganism utilizing the irradiated lignocellulosic material to produce a fuel or other product, e.g., an alcohol, such as ethanol, isobutanol, or n-butanol, a sugar alcohol, such as erythritol, or an organic acid.

In some cases, the lignocellulosic material can include, for example, wood, grasses, e.g., switchgrass, grain residues, e.g., rice hulls, bagasse, jute, hemp, flax, bamboo, sisal, abaca, straw, corn cobs, coconut hair, algae, seaweed, and mixtures of any of these. Cellulosic materials include, for example, paper, paper products, paper pulp, materials having a high α-cellulose content such as cotton, and mixtures of any of these. Any of the methods described herein can be practiced with mixtures of cellulosic and lignocellulosic materials.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a diagrammatic representation of a lignocellulosic material prior to irradiation to reduce its recalcitrance.

FIG. 2 is a diagrammatic representation of the material shown in FIG. 1 after irradiation.

FIG. 3 is a block diagram illustrating conversion of biomass into products and co-products.

FIG. 4 is a block diagram illustrating treatment of biomass and the use of the treated biomass in a fermentation process.

FIGS. 5, 5A and 5B are graphs of electron energy deposition (MeV cm²/g) vs. thickness×density (g/cm²).

### DETAILED DESCRIPTION

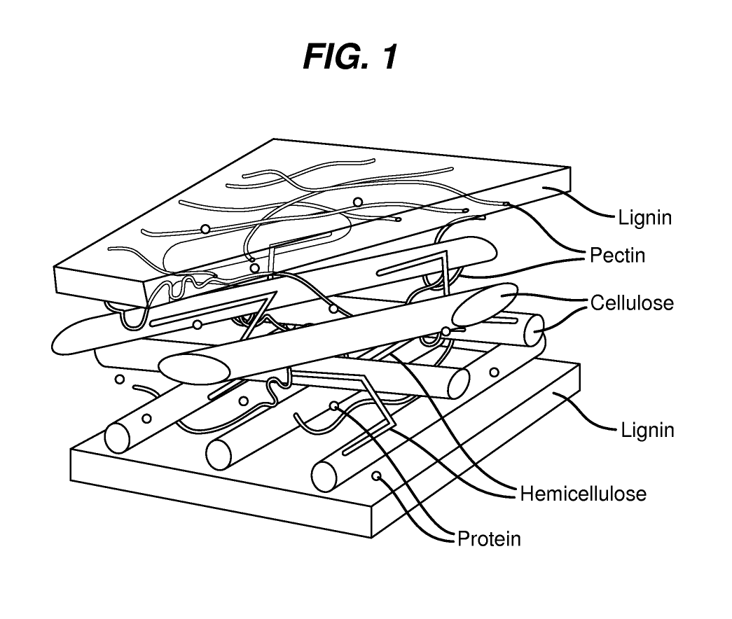

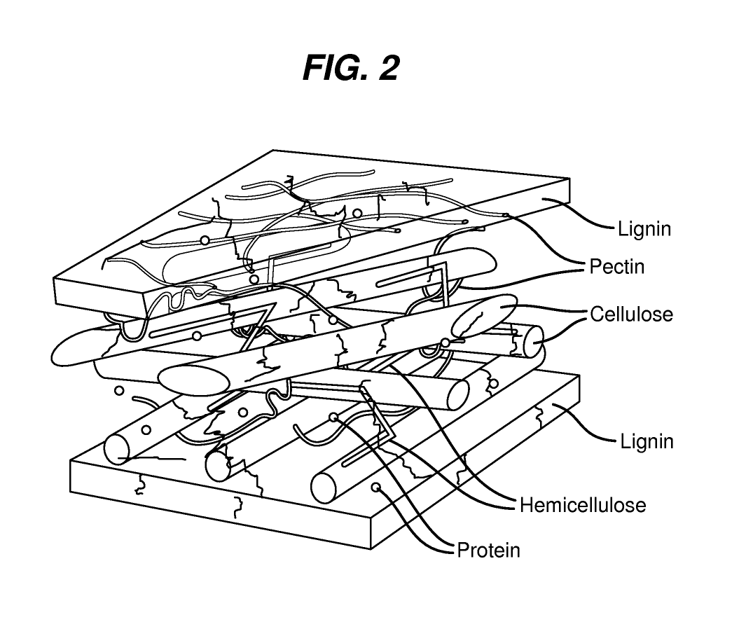

Using the methods described herein, lignocellulosic biomass can be processed to produce fuels and other products, e.g., any of the products described herein. Systems and processes are described below that can use as feedstocks lignocellulosic materials that are readily available, but can be difficult to process by processes such as fermentation. For example, in some cases the feedstock includes corncobs, and for ease of harvesting may include the entire corn plant, including the corn stalk, corn kernels, leaves and roots. To allow such materials to be processed into fuel, the materials are irradiated to reduce their recalcitrance, as shown diagrammatically in FIGS. 1 and 2. As shown diagrammatically in FIG. 2, irradiation causes “fracturing” to occur in the material, disrupting the bonding between lignin, cellulose and hemicellulose that protects the cellulose from enzymatic attack.

In the methods disclosed herein, this irradiating step includes irradiating the lignocellulosic material with relatively low voltage, high power electron beam radiation, often at a relatively high dose rate. Advantageously and ideally, the irradiation equipment is self-shielded (shielded with steel plate rather than by a concrete vault), reliable, electrically efficient, and available commercially. In some cases, the irradiation equipment is greater than 50% electrically efficient, e.g., greater than 60%, 70%, 80%, or even greater than 90% electrically efficient.

The methods further include mechanically treating the starting material, and in some cases the irradiated material. Mechanically treating the material provides a relatively homogeneous, fine material that can be distributed in a thin layer of substantially uniform thickness for irradiation. Mechanical treatment also, in some cases, serves to “open up” the material to enhance its susceptibility to enzymatic attack, and, if performed after irradiation, can increase fracturing of the material and thus further reduce its recalcitrance.

Also discussed herein are enhancements to the saccharification and fermentation processes, including boiling, cooking or steeping the material after irradiation and prior to saccharification.

### Systems for Treating Biomass

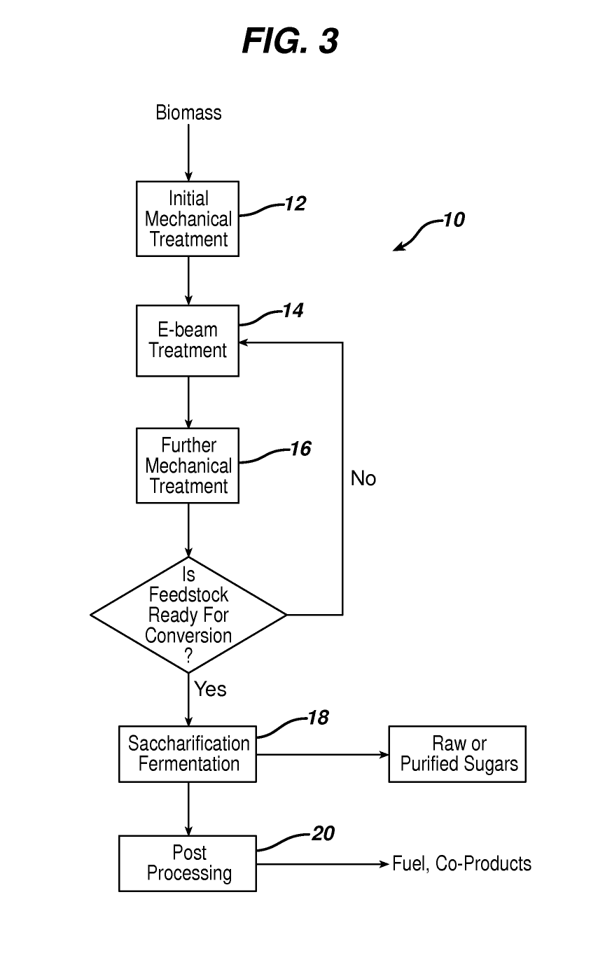

FIG. 3 shows a process 10 for converting biomass, particularly biomass with significant cellulosic and lignocellulosic components, into useful intermediates and products. Process 10 includes initially mechanically treating the feedstock (12), for example by hammermilling, e.g., to reduce the size of the feedstock so that the feedstock can be distributed in a thin, even layer on a conveyor for irradiation by the electron beam. The mechanically treated feedstock is then treated with relatively low voltage, high power electron beam radiation (14) to reduce its recalcitrance, for example by weakening or fracturing bonds in the crystalline structure of the material. The electron beam apparatus may include multiple heads (often called horns), as will be discussed in detail below. Next, the irradiated material is optionally subjected to further mechanical treatment (16). This mechanical treatment can be the same as or different from the initial mechanical treatment. For example, the initial treatment can be a size reduction (e.g., cutting) step followed by a grinding, e.g., hammermilling, or shearing step, while the further treatment can be a grinding or milling step.

The material can then be subjected to further irradiation, and in some cases further mechanical treatment, if further structural change (e.g., reduction in recalcitrance) is desired prior to further processing.

Next, the treated material is saccharified into sugars, and the sugars are fermented (18). If desired, some or all of the sugars can be sold as or incorporated into a product, rather than fermented.

In some cases, the output of step (18) is directly useful but, in other cases, requires further processing provided by a post-processing step (20) to produce a fuel, e.g., ethanol, isobutanol or n-butanol, and in some cases co-products. For example, in the case of an alcohol, post-processing may involve distillation and, in some cases, denaturation.

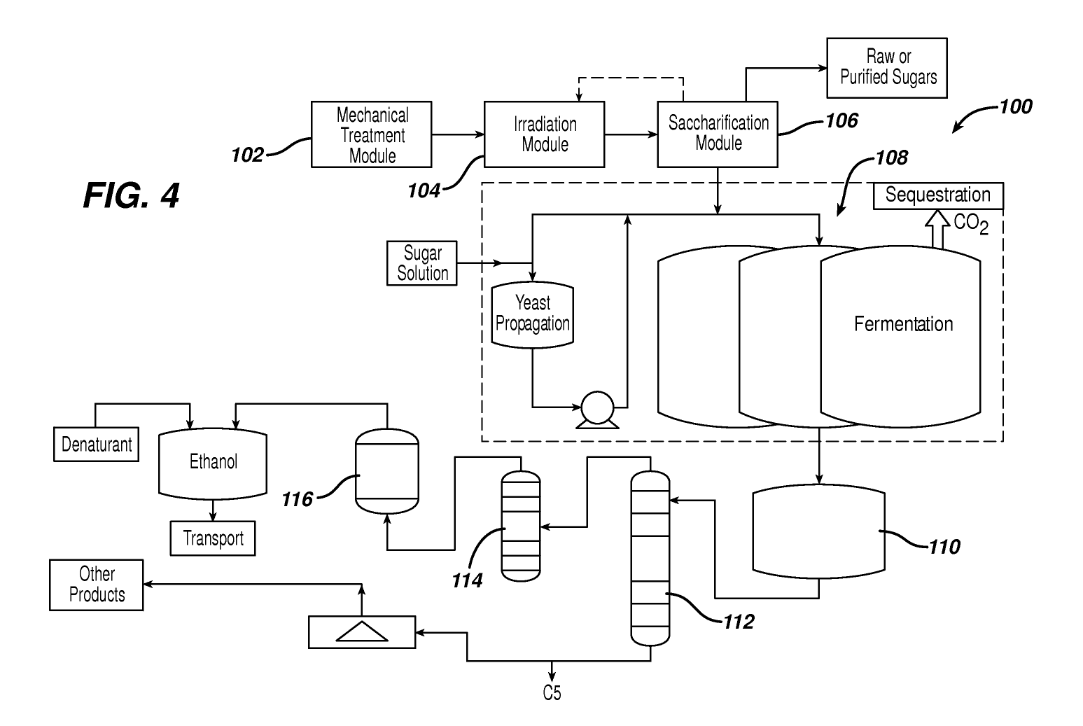

FIG. 4 shows a system 100 that utilizes the steps described above to produce an alcohol. System 100 includes a module 102 in which a biomass feedstock is initially mechanically treated (step 12, above), an electron beam apparatus 104 in which the mechanically treated feedstock is irradiated (step 14, above), and an optional module (not shown) in which the structurally modified feedstock can be subjected to further mechanical treatment (step 16, above). In some implementations the irradiated feedstock is used without further mechanical treatments, while in others it is returned to module 102 for further mechanical treatment rather than being further mechanically treated in a separate module.

After these treatments, which may be repeated as many times as required to obtain desired feedstock properties, the treated feedstock is saccharified into sugars in a saccharification module 106, and the sugars are delivered to a fermentation system 108. In some cases, saccharification and fermentation are performed in a single tank, as discussed in U.S. Ser. No. 61/296,673, the complete disclosure of which is incorporated herein by reference. Mixing may be performed during fermentation, in which case the mixing may be relatively gentle (low shear) so as to minimize damage to shear sensitive ingredients such as enzymes and other microorganisms. In some embodiments, jet mixing is used, as described in U.S. Ser. No. 61/218,832, U.S. Ser. No. 61/179,995 and U.S. Ser. No. 12/782,692, the complete disclosures of which are incorporated herein by reference. In some cases, high shear mixing may be used. In such cases, it is generally desirable to monitor the temperature and/or enzyme activity of the tank contents.

Referring again to FIG. 3, fermentation produces a crude ethanol mixture, which flows into a holding tank 110. Water or other solvent, and other non-ethanol components, are stripped from the crude ethanol mixture using a stripping column 112, and the ethanol is then distilled using a distillation unit 114, e.g., a rectifier. Distillation may be by vacuum distillation. Finally, the ethanol can be dried using a molecular sieve 116 and/or denatured, if necessary, and output to a desired shipping method.

In some cases, the systems described herein, or components thereof, may be portable, so that the system can be transported (e.g., by rail, truck, or marine vessel) from one location to another. The method steps described herein can be performed at one or more locations, and in some cases one or more of the steps can be performed in transit. Such mobile processing is described in U.S. Ser. No. 12/374,549 and International Application No. WO 2008/011598, the full disclosures of which are incorporated herein by reference.

Any or all of the method steps described herein can be performed at ambient temperature. If desired, cooling and/or heating may be employed during certain steps. For example, the feedstock may be cooled during mechanical treatment to increase its brittleness. In some embodiments, cooling is employed before, during or after the initial mechanical treatment and/or the subsequent mechanical treatment. Cooling may be performed as described in Ser. No. 12/502,629, the full disclosure of which is incorporated herein by reference. Moreover, the temperature in the fermentation system 108 may be controlled to enhance saccharification and/or fermentation.

The individual steps of the methods described above, as well as the materials used, will now be described in further detail.

### Mechanical Treatments

Mechanical treatments of the feedstock may include, for example, cutting, milling, e.g., hammermilling, grinding, pressing, shearing or chopping. Suitable hammermills are available from, for example, Bliss Industries, under the tradename ELIMINATOR™ Hammermill, and Schutte-Buffalo Hammermill.

The initial mechanical treatment step may, in some implementations, include reducing the size of the feedstock. In some cases, loose feedstock (e.g., recycled paper or switchgrass) is initially prepared by cutting, shearing and/or shredding. In this initial preparation step screens and/or magnets can be used to remove oversized or undesirable objects such as, for example, rocks or nails from the feed stream.

In addition to this size reduction, which can be performed initially and/or later during processing, mechanical treatment can also be advantageous for “opening up,” “stressing,” breaking or shattering the feedstock materials, making the cellulose of the materials more susceptible to chain scission and/or disruption of crystalline structure during the structural modification treatment. The open materials can also be more susceptible to oxidation when irradiated.

Methods of mechanically treating the feedstock include, for example, milling or grinding. Milling may be performed using, for example, a hammer mill, ball mill, colloid mill, conical or cone mill, disk mill, edge mill, Wiley mill or grist mill. Grinding may be performed using, for example, a cutting/impact type grinder. Specific examples of grinders include stone grinders, pin grinders, coffee grinders, and bun grinders. Grinding or milling may be provided, for example, by a reciprocating pin or other element, as is the case in a pin mill. Other mechanical treatment methods include mechanical ripping or tearing, other methods that apply pressure to the fibers, and air attrition milling. Suitable mechanical treatments further include any other technique that continues the disruption of the internal structure of the material that was initiated by the previous processing steps.

Suitable cutting/impact type grinders include those commercially available from IKA Works under the tradenames A10 Analysis Grinder and M10 Universal Grinder. Such grinders include metal beaters and blades that rotate at high speed (e.g., greater than 30 m/s or even greater than 50 m/s) within a milling chamber. The milling chamber may be at ambient temperature during operation, or may be cooled, e.g., by water or dry ice.

In some implementations, the feedstock, either before or after structural modification, is sheared, e.g., with a rotary knife cutter. The feedstock may also be screened. In some embodiments, the shearing of the feedstock and the passing of the material through a screen are performed concurrently.

### Processing Conditions

The feedstock can be mechanically treated in a dry state, a hydrated state (e.g., having up to 10 percent by weight absorbed water), or in a wet state, e.g., having between about 10 percent and about 75 percent by weight water. In some cases, the feedstock can be mechanically treated under a gas (such as a stream or atmosphere of gas other than air), e.g., oxygen or nitrogen, or steam.

In some cases, the feedstock can be treated as it is being introduced into the reactor in which it will be saccharified, e.g., but injecting steam into or through the material as it is being fed into the reactor.

It is generally preferred that the feedstock be mechanically treated in a substantially dry condition, e.g., having less than 10 percent by weight absorbed water and preferably less than five percent by weight absorbed water) as dry fibers tend to be more brittle and thus easier to structurally disrupt. In a preferred embodiment, a substantially dry, structurally modified feedstock is ground using a cutting/impact type grinder.

However, in some embodiments the feedstock can be dispersed in a liquid and wet milled. The liquid is preferably the liquid medium in which the treated feedstock will be further processed, e.g., saccharified. It is generally preferred that wet milling be concluded before any shear or heat sensitive ingredients, such as enzymes and nutrients, are added to the liquid medium, since wet milling is generally a relatively high shear process. Wet milling can be performed with heat sensitive ingredients, however, as long as the milling time is kept to a minimum, and/or temperature and/or enzyme activity are monitored. In some embodiments, the wet milling equipment includes a rotor/stator arrangement. Wet milling machines include the colloidal and cone mills that are commercially available from IKA Works, Wilmington, N.C. (www.ikausa.com). Wet milling is particularly advantageous when used in combination with the soaking treatments described herein.

If desired, lignin can be removed from any feedstock that includes lignin. Also, to aid in the breakdown of the feedstock, in some embodiments the feedstock can be cooled prior to, during, or after irradiation and/or mechanical treatment, as described in Ser. No. 12/502,629, the full disclosure of which is incorporated herein by reference. In addition, or alternatively, the feedstock can be treated with heat, a chemical (e.g., mineral acid, base or a strong oxidizer such as sodium hypochlorite) and/or an enzyme. However, in many embodiments such additional treatments are unnecessary due to the effective reduction in recalcitrance that is provided by the combination of the mechanical and structure modifying treatments.

### Characteristics of the Mechanically Treated Feedstock

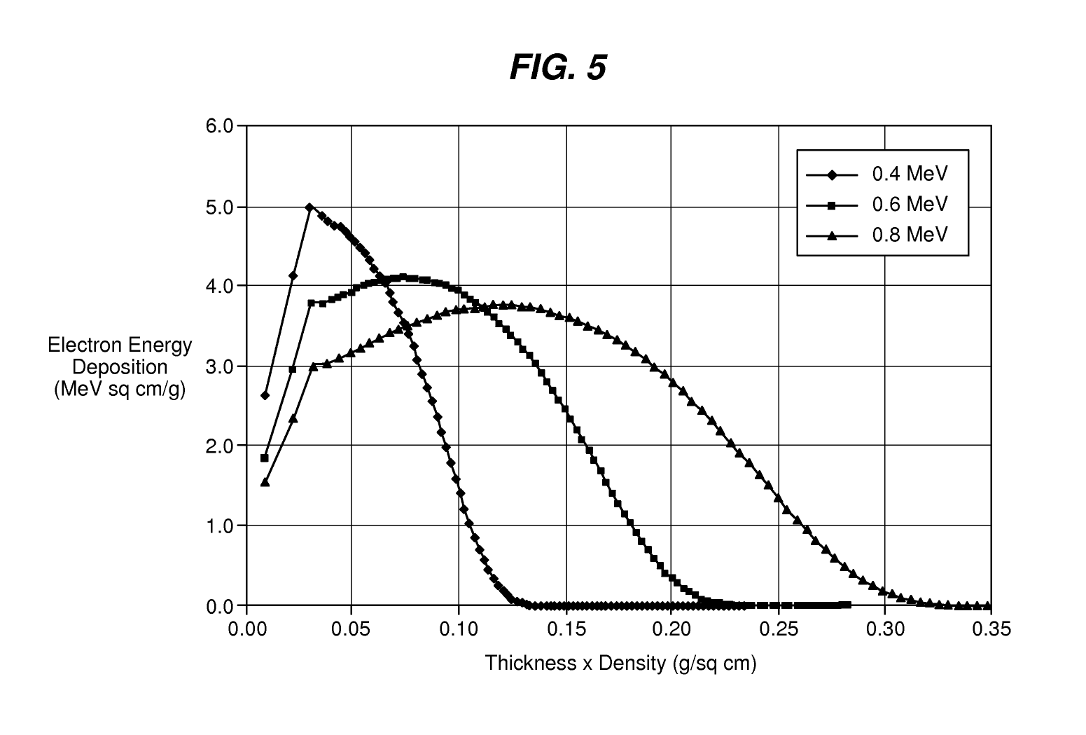

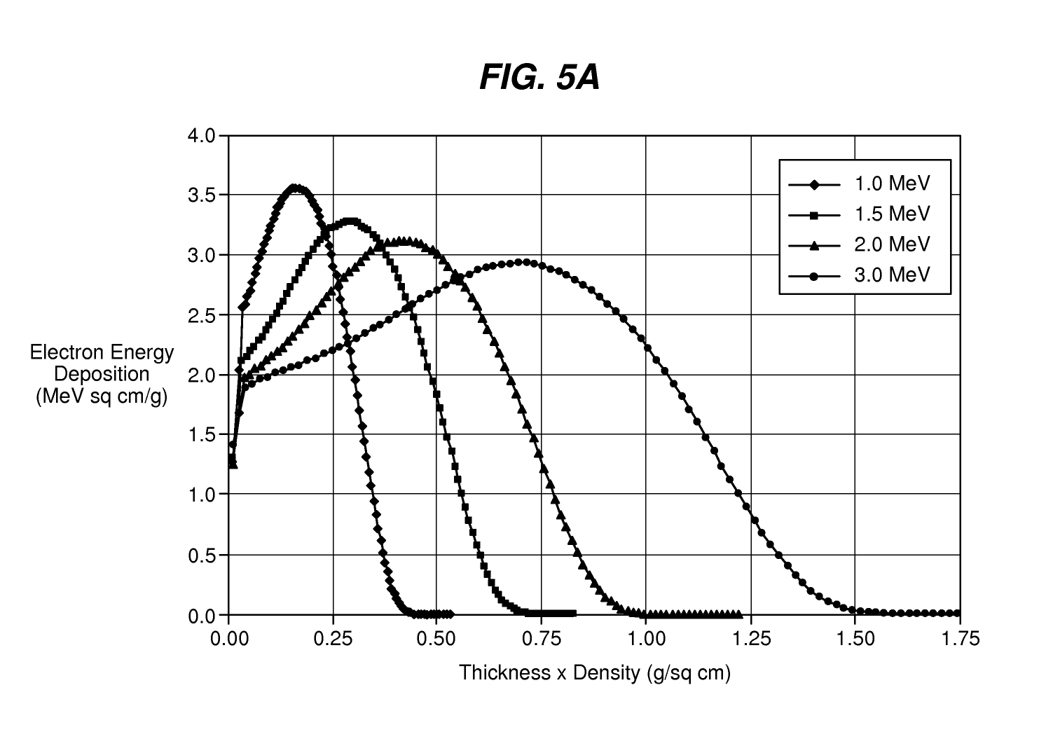

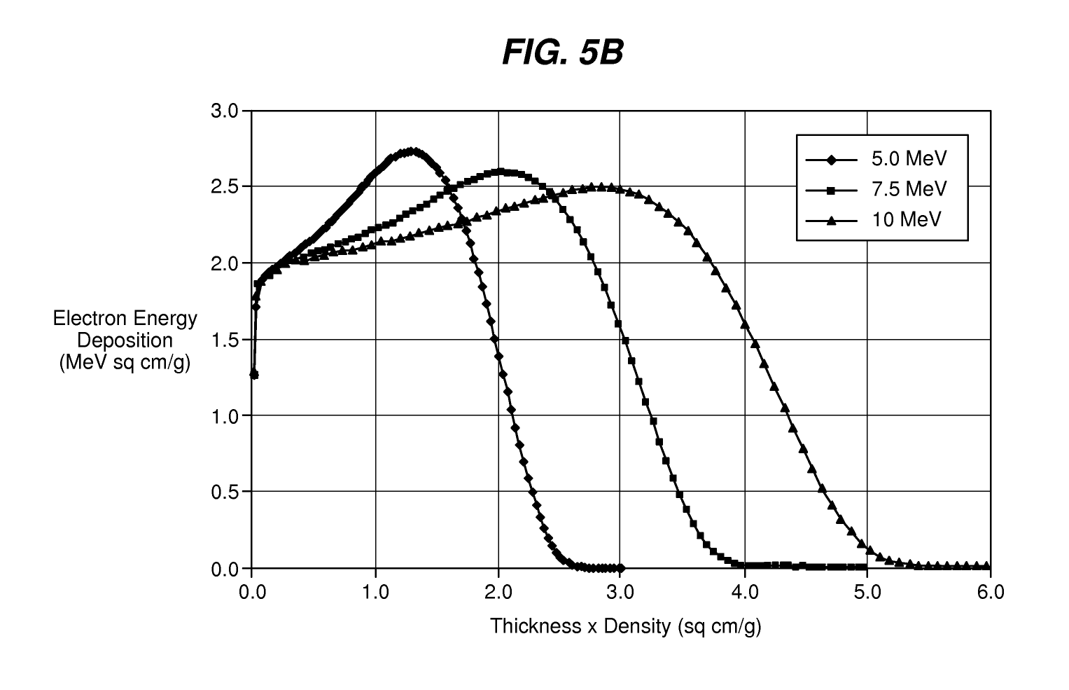

Mechanical treatment systems can be configured to produce feed streams with specific characteristics such as, for example, specific bulk densities, maximum sizes, fiber length-to-width ratios, or surface areas ratios. One desired characteristic of the feedstock is that it is generally homogeneous in size, and of a small enough size so that the feedstock can be transported past the electron beam in a layer of substantially uniform thickness that is less than about 20 mm, e.g., less than 15 mm, less than 10, less than 5, or less than 2 mm, and preferably from about 1 to 10 mm. It is preferred that the standard deviation of the thickness of the layer be less than about 50%, e.g., 10 to 50%, when the voltage is from 3 to 10 MeV. When the voltage is from about 1 to 3 MeV, it is preferred that the standard deviation of the thickness be less than 25%, e.g., from 10 to 25%, and when the voltage is less than 1 MeV it is preferred that the standard deviation be less than 10%. Maintaining the sample thickness within these maximum standard deviations, derived from the data in FIGS. 5-5B, tends to promote dose uniformity within the sample.

It is generally preferred that the particle size of the comminuted feedstock, if it is in particulate form, be relatively small. For example, preferably greater than about 75%, 80%, 85%, 90% or 95% of the feedstock has a particle size of less than about 1.0 mm. It is also desirable that the particle size not be overly fine. For example, in some cases less than about 15%, 10%, 5% or 2% of the feedstock has a particle size of less than about 0.1 mm. In some implementations, the particle size of 75%, 80%, 85%, 90% or 95% of the feedstock is from about 0.25 mm to 2.5 mm, or from about 0.3 mm to 1.0 mm. Generally, it is desirable that the particles not be so large that it is difficult to form a uniform layer of the desired thickness, and not so fine that it is necessary to expend an impractical amount of energy on comminuting the feedstock material.

It is important that the layer be of relatively uniform thickness, and that the material itself be of relatively uniform particle size and density, because of the relationship between material thickness and density and penetration depth of the electron beam. This relationship is particularly important when a relatively low voltage electron beam is used, because the penetration of electron beams in irradiated materials increases linearly with the incident energy of the electrons. As a result, at accelerating voltages of 1 MeV and less there is a marked drop in dosage with increasing penetration depth. With doses of greater than 500 keV the dose tends to increase with depth in the material to about half of the maximum electron range, and then decrease to nearly zero at a greater depth where the electrons have dissipated most of their kinetic energy. Dose uniformity across the sample thickness can be increased by providing a relatively thin sample, as discussed above, controlling the density of the sample (with lower densities being preferred), and applying the radiation in multiple passes rather than a single pass, as will be discussed further below.

Depth-dose distributions in a sample ranging from 0.4 to 10 MeV are shown in FIGS. 5-5B. The shapes of these depth-dose curves can be defined by several useful range parameters. R(opt) is the optimum thickness where the exit dose is equal to the entrance dose. R(50) is the thickness where the exit dose is half of the maximum dose. R(50e) is the thickness where the exit dose is half of the entrance dose. These parameters can be correlated with the incident electron energy E with sufficient accuracy for industrial applications by using the following linear equations:

R(opt)=0.404E−0.161

R(50)=0.435E−0.152

R(50e)=0.458E−0.152

where the electron range values are in g/cm² and the electron energy values are in MeV.

Another important parameter that affects the dose uniformity is the density of the material. Electrons of a given energy will penetrate deeper into a less dense material than a denser one. The mechanical treatments discussed herein are advantageous in that they tend to reduce the bulk density of the feedstock materials. For example, the bulk density of the mechanically treated material may be less than about 0.65 g/cm³, e.g., less than 0.6 g/cm³, less than 0.5 g/cm³, less than 0.35 g/cm³, or even less than 0.20 g/cm³. In some implementations the bulk density is from about 0.25 to 0.65 g/cm³. Bulk density is determined using ASTM D1895B.

Mechanical treatment can also be used to increase the BET surface area and porosity of the material, making the material more susceptible to enzymatic attack.

In some embodiments, a BET surface area of the mechanically treated biomass material is greater than 0.1 m²/g, e.g., greater than 0.25 m²/g, greater than 0.5 m²/g, greater than 1.0 m²/g, greater than 1.5 m²/g, greater than 1.75 m²/g, greater than 5.0 m²/g, greater than 10 m²/g, greater than 25 m²/g, greater than 35 m²/g, greater than 50 m²/g, greater than 60 m²/g, greater than 75 m²/g, greater than 100 m²/g, greater than 150 m²/g, greater than 200 m²/g, or even greater than 250 m²/g.

A porosity of the mechanically treated feedstock, before or after structural modification, can be, e.g., greater than 20 percent, greater than 25 percent, greater than 35 percent, greater than 50 percent, greater than 60 percent, greater than 70 percent, e.g., greater than 80 percent, greater than 85 percent, greater than 90 percent, greater than 92 percent, greater than 94 percent, greater than 95 percent, greater than 97.5 percent, greater than 99 percent, or even greater than 99.5 percent.

The porosity and BET surface area of the material generally increase after each mechanical treatment and after structural modification.

### Electron Beam Treatment

As discussed above, the feedstock is irradiated to modify its structure and thereby reduce its recalcitrance. Irradiation may, for example, reduce the average molecular weight of the feedstock, change the crystalline structure of the feedstock (e.g., by microfracturing within the structure which may or may not alter the crystallinity as measured by diffractive methods), and/or increase the surface area and/or porosity of the feedstock. In some embodiments, structural modification reduces the molecular weight of the feedstock and/or increases the level of oxidation of the feedstock.

Electron beam irradiation provides very high throughput, while the use of a relatively low voltage/high power electron beam device eliminates the need for expensive vault shielding (such devices are “self-shielded”) and provides a safe, efficient process. While the “self-shielded” devices do include shielding (e.g., metal plate shielding), they do not require the construction of a concrete vault, greatly reducing capital expenditure and often allowing an existing manufacturing facility to be used without expensive modification that may tend to decrease the value of the real estate.

Irradiation is performed using an electron beam device that has a nominal energy of less than 10 MeV, e.g., less than 7 MeV, less than 5 MeV, or less than 2 MeV, e.g., from about 0.5 to 1.5 MeV, from about 0.8 to 1.8 MeV, or from about 0.7 to 1 MeV. In some implementations the nominal energy is about 500 to 800 keV.

The electron beam has a relatively high total beam power (the combined beam power of all accelerating heads, or, if multiple accelerators are used, of all accelerators and all heads), e.g., at least 25 kW, e.g., at least 30, 40, 50, 60, 65, 70, 80, 100, 125, or 150 kW. In some cases, the power is even as high as 500 kW, 750 kW, or even 1000 kW or more. In some cases the electron beam has a beam power of 1200 kW or more.

This high total beam power is usually achieved by utilizing multiple accelerating heads. For example, the electron beam device may include two, four, or more accelerating heads. As one example, the electron beam device may include four accelerating heads, each of which has a beam power of 300 kW, for a total beam power of 1200 kW. The use of multiple heads, each of which has a relatively low beam power, prevents excessive temperature rise in the material, thereby preventing burning of the material, and also increases the uniformity of the dose through the thickness of the layer of material.

The temperature increase during irradiation is governed by the following formula:

ΔT=D(ave)/c

where:

ΔT is the adiabatic temperature rise,

D(ave) is the average dose in kGy (J/g), and

c is the thermal capacity in J/g° C.

Thus, there is a balance between irradiating at high doses, which provides good reduction in recalcitrance, and avoiding burning the material, which deleteriously affects the yield of product that can be obtained from the material. By using multiple heads, the material can be irradiated with a relatively low dose per pass, with time between passes for heat to dissipate from the material, while still receiving a relatively high total dose of radiation.

Dose rate is another important factor in the irradiating process. The absorbed dose D is related to the G value (number of molecules or ions produced or destroyed per 100 eV of absorbed ionizing energy) and the molecular weight Mᵣ of the material being irradiated, as expressed by the following equation:

D=Nₐ(100/G)e/Mᵣ

where:

Nₐ is the Avogadro constant (number of molecules/mole),

100/G is the number of electron volts absorbed per reactive molecule,

e is the electron charge in coulombs (also the conversion factor from electron volts to joules), and

Mᵣ represents the mass/mole in grams.

Nₐ=6.022×10²³ and e=1.602×10⁻¹⁹, and thus the above equation can be rewritten as:

D=9.65×10⁶/(MᵣG)

Because molecular weight decreases as a result of irradiation, and the absorbed dose is inversely proportional to molecular weight, as shown above, over time as the material is irradiated an increasing level of radiation energy is required to produce a further incremental decrease in molecular weight. Accordingly, to reduce the energy required by the recalcitrance-reducing process, it is desirable to irradiate as quickly as possible. In general, it is preferred that irradiation be performed at a dose rate of greater than about 0.25 Mrad per second, e.g., greater than about 0.5, 0.75, 1, 1.5, 2, 5, 7, 10, 12, 15, or even greater than about 20 Mrad per second, e.g., about 0.25 to 2 Mrad per second. Higher dose rates generally require higher line speeds, to avoid thermal decomposition of the material. In one implementation, the accelerator is set for 3 MeV, 50 mAmp beam current, and the line speed is 24 feet/minute, for a sample thickness of about 20 mm (comminuted corn cob material with a bulk density of 0.5 g/cm³).

In some implementations, it is desirable to cool the material during irradiation. For example, the material can be cooled while it is being conveyed, for example by a screw extruder or other conveying equipment.

In some embodiments, irradiating is performed until the material receives a total dose of at least 5 Mrad, e.g., at least 10, 20, 30 or at least 40 Mrad. In some embodiments, the irradiating is performed until the material receives a dose of from about 10 Mrad to about 50 Mrad, e.g., from about 20 Mrad to about 40 Mrad, or from about 25 Mrad to about 30 Mrad. In some implementations, a total dose of 25 to 35 Mrad is preferred, applied ideally over a couple of seconds, e.g., at 5 Mrad/pass with each pass being applied for about one second. Applying a dose of greater than 7 to 8 Mrad/pass can in some cases cause thermal degradation of the feedstock material.

Using multiple heads as discussed above, radiation can be applied in multiple passes, for example, two passes at 10 to 20 Mrad/pass, e.g., 12 to 18 Mrad/pass, separated by a few seconds of cool-down, or three passes of 7 to 12 Mrad/pass, e.g., 9 to 11 Mrad/pass. As discussed above, applying the radiation in several relatively low doses, rather than one high dose, tends to prevent overheating of the material and also increases dose uniformity through the thickness of the material. In some implementations, the material is stirred or otherwise mixed during or after each pass and then smoothed into a uniform layer again before the next pass, to further enhance dose uniformity.

In some embodiments, electrons are accelerated to, for example, a speed of greater than 75 percent of the speed of light, e.g., greater than 85, 90, 95, or 99 percent of the speed of light.

In some embodiments, any processing described herein occurs on lignocellulosic material that remains dry as acquired or that has been dried, e.g., using heat and/or reduced pressure. For example, in some embodiments, the cellulosic and/or lignocellulosic material has less than about five percent by weight retained water, measured at 25° C. and at fifty percent relative humidity.

Radiation can be applied while the cellulosic and/or lignocellulosic material is exposed to air, oxygen-enriched air, or even oxygen itself, or blanketed by an inert gas such as nitrogen, argon, or helium. When maximum oxidation is desired, an oxidizing environment is utilized, such as air or oxygen and the distance from the radiation source is optimized to maximize reactive gas formation, e.g., ozone and/or oxides of nitrogen.

Electron beam accelerators are available, for example, from IBA, Belgium, and NHV Corporation, Japan.

Electron beams can be generated, e.g., by electrostatic generators, cascade generators, transformer generators, low energy accelerators with a scanning system, low energy accelerators with a linear cathode, linear accelerators, and pulsed accelerators.

It may be advantageous to provide a double-pass of electron beam irradiation in order to provide a more effective depolymerization process. For example, the feedstock transport device could direct the feedstock (in dry or slurry form) underneath and in a reverse direction to its initial transport direction. Multiple-pass systems can allow a thicker layer of material to be processed and can provide a more uniform irradiation through the thickness of the layer.

The electron beam irradiation device can produce either a fixed beam or a scanning beam. A scanning beam may be advantageous with large scan sweep length and high scan speeds, as this would effectively replace a large, fixed beam width. Further, available sweep widths of 0.5 m, 1 m, 2 m or more are available.

### Sonication, Pyrolysis, Oxidation, Steam Explosion

If desired, one or more sonication, pyrolysis, oxidative, or steam explosion processes can be used in addition to irradiation to further structurally modify the mechanically treated feedstock. These processes are described in detail in U.S. Ser. No. 12/429,045, the full disclosure of which is incorporated herein by reference.

### Saccharification and Fermentation

### Saccharification

In order to convert the treated feedstock to a form that can be readily fermented, in some implementations the cellulose in the feedstock is first hydrolyzed to low molecular weight carbohydrates, such as sugars, by a saccharifying agent, e.g., an enzyme, a process referred to as saccharification. The irradiated lignocellulosic material that includes the cellulose is treated with the enzyme, e.g., by combining the material and the enzyme in a medium, e.g., in an aqueous solution. As discussed above, preferably jet mixing is used to agitate the mixture of lignocellulosic material, medium, and enzyme during saccharification.

In some cases, the irradiated material is boiled, steeped, or cooked in hot water prior to saccharification. Preferably, the irradiated material is soaked in water at a temperature of about 50° C. to 100° C., preferably about 70° C. to 100° C. Soaking (e.g., boiling or steeping) can be performed for any desired time, for example about 10 minutes to 2 hours, preferably 30 min to 1.5 hours, e.g., 45 min to 75 min. In some implementations the soaking time is at least 2 hours, or at least 6 hours. Generally, the time will be shorter the higher the temperature of the water.

It is not necessary to add any swelling agents or other additives to the water, and in fact doing so will increase cost and may in some cases have a deleterious effect on further processing, if the additive is harmful to the microorganisms used in saccharification and/or fermentation.

Generally, soaking is performed at ambient pressure, for simplicity of processing. However, if desired the mixture of water and irradiated material may be processed under elevated pressure, e.g., under pressure cooker conditions.

After soaking, the mixture is cooled or allowed to cool until a suitable temperature for fermentation is reached, e.g., about 30° C. for yeasts or about 37° C. for bacteria.

### Fermentation

After saccharification, the sugars produced by the saccharification process are fermented to produce, e.g., alcohol(s), sugar alcohols, such as erythritol, or organic acids, e.g., lactic, glutamic or citric acids or amino acids. Yeast and Zymomonas bacteria, for example, can be used for fermentation. Other microorganisms are discussed in the Materials section, below.

The optimum pH for yeast is from about pH 4 to 5, while the optimum pH for Zymomonas is from about pH 5 to 6. Typical fermentation times are about 24 to 96 hours with temperatures in the range of 26° C. to 40° C., however thermophilic microorganisms prefer higher temperatures.

As discussed above, jet mixing may be used during fermentation, and in some cases saccharification and fermentation are performed in the same tank.

Nutrients may be added during saccharification and/or fermentation, for example the food-based nutrient packages described in U.S. Ser. No. 61/365,493, the complete disclosure of which is incorporated herein by reference.

Mobile fermentors can be utilized, as described in U.S. Ser. No. 12/374,549 and International Application No. WO 2008/011598. Similarly, the saccharification equipment can be mobile. Further, saccharification and/or fermentation may be performed in part or entirely during transit.

### Post-Processing

### Distillation

After fermentation, the resulting fluids can be distilled using, for example, a “beer column” to separate ethanol and other alcohols from the majority of water and residual solids. The vapor exiting the beer column can be, e.g., 35% by weight ethanol and can be fed to a rectification column. A mixture of nearly azeotropic (92.5%) ethanol and water from the rectification column can be purified to pure (99.5%) ethanol using vapor-phase molecular sieves. The beer column bottoms can be sent to the first effect of a three-effect evaporator. The rectification column reflux condenser can provide heat for this first effect. After the first effect, solids can be separated using a centrifuge and dried in a rotary dryer. A portion (25%) of the centrifuge effluent can be recycled to fermentation and the rest sent to the second and third evaporator effects. Most of the evaporator condensate can be returned to the process as fairly clean condensate with a small portion split off to waste water treatment to prevent build-up of low-boiling compounds.

### Intermediates and Products

Specific examples of products that may be produced utilizing the processes disclosed herein include, but are not limited to, hydrogen, alcohols (e.g., monohydric alcohols or dihydric alcohols, such as ethanol, n-propanol or n-butanol), sugars, e.g., glucose, xylose, arabinose, mannose, galactose, and mixtures thereof, biodiesel, organic acids (e.g., acetic acid, citric acid, glutamic acid, and/or lactic acid), hydrocarbons, co-products (e.g., proteins, such as cellulolytic proteins (enzymes) or single cell proteins), and mixtures of any of these. Other examples include carboxylic acids, such as acetic acid or butyric acid, salts of a carboxylic acid, a mixture of carboxylic acids and salts of carboxylic acids and esters of carboxylic acids (e.g., methyl, ethyl and n-propyl esters), ketones, aldehydes, alpha, beta unsaturated acids, such as acrylic acid and olefins, such as ethylene. Other alcohols and alcohol derivatives include propanol, propylene glycol, 1,4-butanediol, 1,3-propanediol, methyl or ethyl esters of any of these alcohols. Other products include sugar alcohols, e.g., erythritol, methyl acrylate, methylmethacrylate, lactic acid, propionic acid, butyric acid, succinic acid, 3-hydroxypropionic acid, a salt of any of the acids and a mixture of any of the acids and respective salts.

Any combination of the above products with each other, and/or of the above products with other products, which other products may be made by the processes described herein or otherwise, may be packaged together and sold as products. The products may be combined, e.g., mixed, blended or co-dissolved, or may simply be packaged or sold together.

Any of the products or combinations of products described herein may be irradiated prior to selling the products, e.g., after purification or isolation or even after packaging, for example to sanitize or sterilize the product(s) and/or to neutralize one or more potentially undesirable contaminants that could be present in the product(s). Such irradiation may, for example, be at a dosage of less than about 20 Mrad, e.g., from about 0.1 to 15 Mrad, from about 0.5 to 7 Mrad, or from about 1 to 3 Mrad.

The processes described herein can produce various by-product streams useful for generating steam and electricity to be used in other parts of the plant (co-generation) or sold on the open market. For example, steam generated from burning by-product streams can be used in a distillation process. As another example, electricity generated from burning by-product streams can be used to power electron beam generators used in pretreatment.

The by-products used to generate steam and electricity are derived from a number of sources throughout the process. For example, anaerobic digestion of wastewater can produce a biogas high in methane and a small amount of waste biomass (sludge). As another example, post-saccharification and/or post-distillate solids (e.g., unconverted lignin, cellulose, and hemicellulose remaining from the pretreatment and primary processes) can be used, e.g., burned, as a fuel.

### Materials

### Feedstock Materials

The feedstock is preferably a lignocellulosic material, although the processes described herein may also be used with cellulosic materials, e.g., paper, paper products, paper pulp, cotton, and mixtures of any of these, and other types of biomass. The processes described herein are particularly useful with lignocellulosic materials, because these processes are particularly effective in reducing the recalcitrance of lignocellulosic materials and allowing such materials to be processed into products and intermediates in an economically viable manner.

In some cases, the lignocellulosic material can include, for example, wood, grasses, e.g., switchgrass, grain residues, e.g., rice hulls, bagasse, jute, hemp, flax, bamboo, sisal, abaca, straw, corn cobs, corn stover, coconut hair, algae, seaweed, and mixtures of any of these.

In some cases, the lignocellulosic material includes corncobs. Ground or hammermilled corncobs can be spread in a layer of relatively uniform thickness for irradiation, and after irradiation are easy to disperse in the medium for further processing. To facilitate harvest and collection, in some cases the entire corn plant is used, including the corn stalk, corn kernels, and in some cases even the root system of the plant.

Advantageously, no additional nutrients (other than a nitrogen source, e.g., urea or ammonia) are required during fermentation of corncobs or feedstocks containing significant amounts of corncobs.

Corncobs, before and after comminution, are also easier to convey and disperse, and have a lesser tendency to form explosive mixtures in air than other feedstocks such as hay and grasses.

Other biomass feedstocks include starchy materials and microbial materials.

In some embodiments, the biomass material includes a carbohydrate that is or includes a material having one or more β-1,4-linkages and having a number average molecular weight between about 3,000 and 50,000. Such a carbohydrate is or includes cellulose (I), which is derived from (β-glucose 1) through condensation of β(1,4)-glycosidic bonds. This linkage contrasts itself with that for α(1,4)-glycosidic bonds present in starch and other carbohydrates.

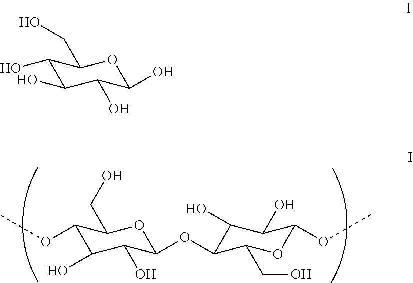

Starchy materials include starch itself, e.g., corn starch, wheat starch, potato starch or rice starch, a derivative of starch, or a material that includes starch, such as an edible food product or a crop. For example, the starchy material can be arracacha, buckwheat, banana, barley, cassava, kudzu, oca, sago, sorghum, regular household potatoes, sweet potato, taro, yams, or one or more beans, such as favas, lentils or peas. Blends of any two or more starchy materials are also starchy materials.

In some cases the biomass is a microbial material. Microbial sources include, but are not limited to, any naturally occurring or genetically modified microorganism or organism that contains or is capable of providing a source of carbohydrates (e.g., cellulose), for example, protists, e.g., animal protists (e.g., protozoa such as flagellates, amoeboids, ciliates, and sporozoa) and plant protists (e.g., algae such alveolates, chlorarachniophytes, cryptomonads, euglenids, glaucophytes, haptophytes, red algae, stramenopiles, and viridaeplantae). Other examples include seaweed, plankton (e.g., macroplankton, mesoplankton, microplankton, nanoplankton, picoplankton, and femptoplankton), phytoplankton, bacteria (e.g., gram positive bacteria, gram negative bacteria, and extremophiles), yeast and/or mixtures of these. In some instances, microbial biomass can be obtained from natural sources, e.g., the ocean, lakes, bodies of water, e.g., salt water or fresh water, or on land. Alternatively or in addition, microbial biomass can be obtained from culture systems, e.g., large scale dry and wet culture systems.

Blends of any biomass materials described herein can be utilized for making any of the intermediates or products described herein. For example, blends of cellulosic materials and starchy materials can be utilized for making any product described herein

### Saccharifying Agents

Cellulases are capable of degrading biomass, and may be of fungal or bacterial origin. Suitable enzymes include cellulases from the genera Bacillus, Pseudomonas, Humicola, Fusarium, Thielavia, Acremonium, Chrysosporium and Trichoderma, and include species of Humicola, Coprinus, Thielavia, Fusarium, Myceliophthora, Acremonium, Cephalosporium, Scytalidium, Penicillium or Aspergillus (see, e.g., EP 458162), especially those produced by a strain selected from the species Humicola insolens (reclassified as Scytalidium thermophilum, see, e.g., U.S. Pat. No. 4,435,307), Coprinus cinereus, Fusarium oxysporum, Myceliophthora thermophila, Meripilus giganteus, Thielavia terrestris, Acremonium sp., Acremonium persicinum, Acremonium acremonium, Acremonium brachypenium, Acremonium dichromosporum, Acremonium obclavatum, Acremonium pinkertoniae, Acremonium roseogriseum, Acremonium incoloratum, and Acremonium furatum; preferably from the species Humicola insolens DSM 1800, Fusarium oxysporum DSM 2672, Myceliophthora thermophila CBS 117.65, Cephalosporium sp. RYM-202, Acremonium sp. CBS 478.94, Acremonium sp. CBS 265.95, Acremonium persicinum CBS 169.65, Acremonium acremonium AHU 9519, Cephalosporium sp. CBS 535.71, Acremonium brachypenium CBS 866.73, Acremonium dichromosporum CBS 683.73, Acremonium obclavatum CBS 311.74, Acremonium pinkertoniae CBS 157.70, Acremonium roseogriseum CBS 134.56, Acremonium incoloratum CBS 146.62, and Acremonium furatum CBS 299.70H. Cellulolytic enzymes may also be obtained from Chrysosporium, preferably a strain of Chrysosporium lucknowense. Additionally, Trichoderma (particularly Trichoderma viride, Trichoderma reesei, and Trichoderma koningii), alkalophilic Bacillus (see, for example, U.S. Pat. No. 3,844,890 and EP 458162), and Streptomyces (see, e.g., EP 458162) may be used.

### Fermentation Agents

The microorganism(s) used in fermentation can be natural microorganisms and/or engineered microorganisms. For example, the microorganism can be a bacterium, e.g., a cellulolytic bacterium, a fungus, e.g., a yeast, a plant or a protist, e.g., an algae, a protozoa or a fungus-like protist, e.g., a slime mold. When the organisms are compatible, mixtures of organisms can be utilized.

Suitable fermenting microorganisms have the ability to convert carbohydrates, such as glucose, xylose, arabinose, mannose, galactose, oligosaccharides or polysaccharides into fermentation products. Fermenting microorganisms include strains of the genus Sacchromyces spp. e.g., Sacchromyces cerevisiae (baker's yeast), Saccharomyces distaticus, Saccharomyces uvarum; the genus Kluyveromyces, e.g., species Kluyveromyces marxianus, Kluyveromyces fragilis; the genus Candida, e.g., Candida pseudotropicalis, and Candida brassicae, Pichia stipitis (a relative of Candida shehatae, the genus Clavispora, e.g., species Clavispora lusitaniae and Clavispora opuntiae the genus Pachysolen, e.g., species Pachysolen tannophilus, the genus Bretannomyces, e.g., species Bretannomyces clausenii (Philippidis, G. P., 1996, Cellulose bioconversion technology, in Handbook on Bioethanol: Production and Utilization, Wyman, C. E., ed., Taylor & Francis, Washington, D.C., 179-212).

Commercially available yeasts include, for example, Red Star®/Lesaffre Ethanol Red (available from Red Star/Lesaffre, USA) FALI® (available from Fleischmann's Yeast, a division of Burns Philip Food Inc., USA), SUPERSTART® (available from Alltech, now Lalemand), GERT STRAND® (available from Gert Strand AB, Sweden) and FERMOL® (available from DSM Specialties). Yeasts such as Moniliella pollinis may be used to produce sugar alcohols such as erythritol.

Bacteria may also be used in fermentation, e.g., Zymomonas mobilis and Clostridium thermocellum (Philippidis, 1996, supra).

### Other Embodiments

A number of embodiments of the invention have been described. Nevertheless, it will be understood that various modifications may be made without departing from the spirit and scope of the invention.

For example, the process parameters of any of the processing steps discussed herein can be adjusted based on the lignin content of the feedstock, for example as disclosed in U.S. Provisional Application No. 61/151,724, and U.S. Ser. No. 12/704,519, the full disclosures of which are incorporated herein by reference.

Also, the processes described herein can be used to manufacture a wide variety of products and intermediates, in addition to or instead of sugars and alcohols. Intermediates or products that can be manufactured using the processes described herein include energy, fuels, foods and materials. Specific examples of products include, but are not limited to, hydrogen, alcohols (e.g., monohydric alcohols or dihydric alcohols, such as ethanol, n-propanol or n-butanol), hydrated or hydrous alcohols, e.g., containing greater than 10%, 20%, 30% or even greater than 40% water, xylitol, sugars, biodiesel, organic acids (e.g., acetic acid and/or lactic acid), hydrocarbons, co-products (e.g., proteins, such as cellulolytic proteins (enzymes) or single cell proteins), and mixtures of 1 0 any of these in any combination or relative concentration, and optionally in combination with any additives, e.g., fuel additives. Other examples include carboxylic acids, such as acetic acid or butyric acid, salts of a carboxylic acid, a mixture of carboxylic acids and salts of carboxylic acids and esters of carboxylic acids (e.g., methyl, ethyl and n-propyl esters), ketones (e.g., acetone), aldehydes (e.g., acetaldehyde), alpha, beta unsaturated acids, such as acrylic acid and olefins, such as ethylene. Other alcohols and alcohol derivatives include propanol, propylene glycol, 1,4-butanediol, 1,3-propanediol, methyl or ethyl esters of any of these alcohols. Other products include methyl acrylate, methylmethacrylate, lactic acid, propionic acid, butyric acid, succinic acid, 3-hydroxypropionic acid, a salt of any of the acids, and a mixture of any of the acids and respective salts.

Other intermediates and products, including food and pharmaceutical products, are described in U.S. Ser. No. 12/417,900, the full disclosure of which is hereby incorporated by reference herein.

Accordingly, other embodiments are within the scope of the following claims.

What is claims is:

1. A method comprising:
steeping an irradiated lignocellulosic material in water; and
saccharifying the steeped irradiated lignocellulosic material.

2. The method of claim 1 wherein steeping the irradiated lignocellulosic material is at a temperature of at least 40° C.

3. The method of claim 1 wherein steeping the irradiated lignocellulosic material is at a temperature between about 70° C. to 100° C.

4. The method of claim 1 wherein steeping the irradiated lignocellulosic material is at an elevated pressure.

5. The method of claim 1 wherein steeping the irradiated lignocellulosic material is for 10 minutes to about 2 hours.

6. The method of claim 1 wherein steeping is for at least 6 hours.

7. The method of claim 1 further comprising treating the irradiated lignocellulosic material with a mineral acid.

8. The method of claim 1 wherein saccharifying includes treating lignocellulosic material with an enzyme.

9. The method of claim 1 wherein the lignocellulosic material is irradiated with an electron beam at a voltage of less than 3 MeV and a power of at least 60 kW.

10. The method of claim 9 wherein the electron beam operates at a voltage of less than 1 MeV.

11. The method of claim 9 wherein the electron beam operates at a power of at least 100 kW.

12. The method of claim 9 wherein the lignocellulosic material receives a total dosage of between 25 and 35 Mrads.

13. The method of claim 9 wherein irraditating the material includes multiple passes of irradiation with each pass delivering a dose of 20 Mrad or less.

14. The method of claim 1 further comprising combining the lignocellulosic material with an organism.

15. The method of claim 14 wherein the organism is a sugar fermenting organism.

16. The method of claim 1 further comprising hammer milling the lignocellulosic material prior to irradiating the lignocellulosic material.

17. The method of claim 1 wherein the lignocellulosic material is selected from the group consisting of wood, grasses, switchgrass, grain residues, rice hulls, bagasse, jute, hemp, flax, bamboo, sisal, abaca, straw, corn cobs, corn stover, coconut hair, algae, seaweed, and mixtures of any of these.

18. A method comprising:
treating and saccharifying a lignocellulosic material, wherein
treating includes steeping in water an electron beam irradiated lignocellulosic material.

19. The method of claim 18 wherein steeping is at a temperature of at least 40° C.

20. The method of claim 18 wherein steeping is at a temperature between about 70° C. to 100° C.

21. The method of claim 18 wherein steeping is at an elevated pressure.

22. The method of claim 18 wherein steeping is for 10 minutes to about 2 hours.

23. The method of claim 18 wherein steeping is for at least 6 hours.

24. The method of claim 18 wherein saccharifying includes treating the lignocellulosic material with an enzyme.

25. The method of claim 18 wherein treating includes treating with a mineral acid.
